(19) United States
(12) Patent Application Publication
(10) Pub. No.: US 2024/0225642 A1
(43) Pub. Date: Jul. 11, 2024

(54) SURGICAL STAPLING DEVICE WITH STOPPER FOR PUSHER/KNIFE RETENTION

(71) Applicant: Covidien LP, Mansfield, MA (US)

(72) Inventors: Ping Ren, Shanghai (CN); Xiliang Zhang, Shanghai (CN)

(21) Appl. No.: 18/558,783

(22) PCT Filed: May 7, 2021
(86) PCT No.: PCT/CN2021/092065

§ 371 Date: Nov. 3, 2023

(51) Int. Cl. A61B 17/072

(52) U.S. Cl. CPC A61B 17/072; A61B 2017/07221; A61B 2017/07271; A61B 2017/07285

(57) ABSTRACT

A surgical stapling device includes an end effector that has a cartridge assembly with a stopper of simple construction that prevents advancement of a pusher and a knife assembly within a cartridge body of the cartridge assembly until the stapling device is fired, and/or prevents readvancement of the knife assembly of the cartridge assembly after the stapling device is fired.

### FIELD

This technology is generally related to surgical stapling devices and, more particularly, to surgical stapling devices with replaceable or removable cartridge assemblies.

### BACKGROUND

Surgical stapling devices are commonly used during a variety of surgical procedures to expedite dissection and suturing of tissue and to minimize trauma to a patient. Typically, these stapling devices include an end effector having a cartridge assembly and an anvil assembly that are movable in relation to each other between open and clamped positions. The cartridge assembly is configured to be removable from the end effector and replaceable after each use of the stapling device to facilitate reuse of the stapling device. The cartridge assembly typically includes a knife assembly for cutting tissue that is clamped between the anvil and cartridge assemblies.

Typically, the cartridge assembly includes a cartridge body, a pusher, a knife assembly, and staples. The cartridge body defines a cavity and staple receiving slots. The pusher and the knife assembly are received within the cavity of the cartridge body and are movable between retracted and advanced positions to eject the staples from the staple receiving slots and to advance a cutting blade of a knife assembly from a position recessed within the cartridge body to a position extending from the cartridge body.

It is known in the art to include a stop member on the cartridge body to retain the pusher and the knife assembly in their retracted positions until the cartridge body is loaded onto the end effector and fired. The stop member prevents the pusher and knife assembly from advancing within the cartridge body prior to firing of the stapling device. Premature advancement of the pusher and/or knife assembly within the cartridge body results in ejection of staples from the cartridge body and/or unwanted exposure the cutting blade.

It is also known to provide a stopper on the cartridge body to prevent readvancement of the knife assembly after the stapling device is fired. This may occur during transport or disposal of the spent cartridge assembly. Readvancement of the knife assembly results in unwanted exposure of the knife which may cause injury to a clinician.

A continuing need exists in the art for a surgical stapling device that includes a replaceable cartridge assembly that has a simple but effective mechanism for retaining the pusher and knife assembly in a retracted position prior to firing of the stapling device and for retaining the knife assembly in a retracted position after the stapling device has been fired.

### SUMMARY

This disclosure is directed to a surgical stapling device that includes an end effector that has a cartridge assembly with a stopper of simple construction that prevents advancement of a pusher and a knife assembly within a cartridge body of the cartridge assembly until the stapling device is fired. This disclosure is also directed to a stapling device that includes a stopper of simple construction that prevents readvancement of the knife assembly of the cartridge assembly after the stapling device is fired.

Aspects of this disclosure are directed to a cartridge assembly including a cartridge body, staples, a pusher, a knife assembly, and a resilient stopper. The cartridge body defines a cavity, a knife slot that communicates with the cavity, and a plurality of staple receiving slots. The staple receiving slots are positioned on opposite sides of the knife slot. One of the staples is supported within each of the staple receiving slots of the cartridge body. The pusher is supported within the cavity of the cartridge body and includes a base portion and fingers that extend distally from the base portion into the staple receiving slots. The base portion defines a knife receiving slot. The knife assembly includes a knife holder and a cutting blade that extends distally from the knife holder. The cutting blade extends through the knife receiving slot of the base portion of the pusher and is movable from a retracted position to an advanced position to move the cutting blade through the knife slot of the cartridge body. The resilient stopper is supported on the cartridge body and is deformable from a first position in which the resilient stopper extends into the cavity of the cartridge body to obstruct movement of the pusher and the knife assembly from their retracted positions to their advanced positions to a second position allowing movement of the pusher and the knife assembly from their retracted positions to their advanced positions.

In aspects of the disclosure, the cartridge body includes a side wall that defines a recess and an opening that communicates with the recess and the cavity, and the resilient stopper is positioned within the recess.

In some aspects of the disclosure, the stopper is in a non-deformed position in the first position.

In certain aspects of the disclosure, the pusher includes an abutment member that is positioned to engage the stopper to deform the stopper from the first position to the second position.

In aspects of the disclosure, the abutment member includes a ramped distal surface that is positioned to engage the stopper to move the stopper from the first position to the second position.

In some aspects of the disclosure, the stopper includes a base portion and an arm, and the base portion is fixedly secured within the recess and the arm is coupled to the base portion in cantilevered fashion.

In certain aspects of the disclosure, the arm has a proximally facing stop surface that is positioned within the cavity of the cartridge body when the stopper is in the first position.

In aspects of the disclosure, the proximally facing stop surface is axially aligned with the abutment member of the pusher.

In some aspects of the disclosure, the knife holder includes an engagement member that is axially aligned with the abutment member of the pusher and the proximally facing stop surface of the arm.

In certain aspects of the disclosure, the arm includes a distally facing cam surface, and the engagement member is axially aligned with the distally facing cam surface and movable into engagement with the distally facing cam surface when the knife holder is retracted to move the stopper to the second position and allow the knife holder to move from the advanced position back to the retracted position.

In aspects of the disclosure, the stopper moves back to the first position after the knife holder moves back to its retracted position to prevent readvancement of the knife holder.

In some aspects of the disclosure, the stopper is positioned to engage the pusher to frictionally retain the pusher in its retracted position.

In certain aspects of the disclosure, the stopper includes a resilient arm that includes a distally facing cam surface and a proximal stop surface.

In aspects of the disclosure, the knife holder is movable into engagement with the distally facing cam surface of the resilient arm as the knife holder is moved from its advanced position towards its retracted position to move the resilient arm of the stopper from the first position to the second position and allow the knife holder to return to its retracted position.

In some aspects of the disclosure, the resilient arm of the stopper moves back to the first position after the knife holder moves back to its retracted position.

In certain aspects of the disclosure, the proximal stop surface of the resilient arm of the stopper is positioned to prevent readvancement of the knife holder when the stopper is in the first position.

In aspects of the disclosure, the stopper is secured to an inner surface of the side wall of the cartridge body and includes a resilient arm that is angled into the cavity in a proximal direction.

In some aspects of the disclosure, the resilient arm of the stopper is movable from the first position to the second position in response to movement of the pusher from its retracted position towards its advanced position.

In certain aspects of the disclosure, the pusher includes an abutment member that has a ramped distal surface that is positioned to engage the resilient arm to move the resilient arm from the first position to the second position.

Other aspects of the disclosure are directed to a surgical stapling device including an elongate body, and an end effector. The elongate body has a proximal portion and a distal portion. The end effector is supported on the distal portion of the elongate body and includes an end effector frame and a reload assembly. The reload assembly includes a reload frame, an anvil assembly, and a cartridge assembly. The anvil assembly is supported on the reload frame. The cartridge assembly is supported on the reload frame and includes a cartridge body, staples, a pusher, a knife assembly, and a resilient stopper. The cartridge body defines a cavity, a knife slot that communicates with the cavity, and a plurality of staple receiving slots. The staple receiving slots are positioned on opposite sides of the knife slot. One of the staples is supported within each of the staple receiving slots of the cartridge body. The pusher is supported within the cavity of the cartridge body and includes a base portion and fingers extending distally from the base portion into the staple receiving slots. The base portion defines a knife receiving slot. The knife assembly includes a knife holder and a cutting blade that extends distally from the knife holder. The cutting blade extends through the knife receiving slot of the base portion of the pusher and is movable from a retracted position to an advanced position to move the cutting blade through the knife slot of the cartridge body. The resilient stopper is supported on the cartridge body and is deformable between a first position extending into the cavity of the cartridge body to obstruct movement of the pusher and the knife assembly from their retracted positions to their advanced positions and a second position allowing movement of the pusher and the knife assembly from their retracted positions to their advanced positions.

In aspects of the disclosure, the stapling device includes a clamp slide assembly and a thrust bar.

In some aspects of the disclosure, the clamp slide assembly defines a cavity that removably receives the cartridge assembly of the end effector.

In certain aspects of the disclosure, the clamp slide assembly is movable between retracted and advanced positions to move the cartridge assembly in relation to the anvil assembly between open and clamped positions.

In aspects of the disclosure, the thrust bar is adapted to releasably engage the knife holder of the cartridge assembly and is movable from a retracted position to an advanced position to advance the pusher and eject the staples from the cartridge body of the cartridge assembly.

Other aspects of the disclosure are directed to a cartridge assembly including a cartridge body, staples, a pusher, a knife assembly, and a resilient stopper. The cartridge body defines a cavity, a knife slot that communicates with the cavity, and a plurality of staple receiving slots. The staple receiving slots are positioned on opposite sides of the knife slot. One of the staples is supported within each of the staple receiving slots of the cartridge body. The pusher is supported within the cavity of the cartridge body and defines a knife receiving slot. The knife assembly includes a knife holder and a cutting blade that extends distally from the knife holder. The cutting blade is movable from a retracted position to an advanced position to move the cutting blade through the knife slot of the cartridge body. The stopper is supported on the cartridge body and is movable between a first position extending into the cavity of the cartridge body to obstruct movement of the pusher and the knife assembly from their retracted positions to their advanced positions and a second position allowing movement of the pusher and the knife assembly from their retracted positions to their advanced positions.

Other features of the disclosure will be appreciated from the following description.

### BRIEF DESCRIPTION OF THE DRAWINGS

Various aspects of the disclosure are described herein below with reference to the drawings, wherein:

FIG. 1 is a side perspective view of a surgical stapling device according to aspects of the disclosure including an end effector in an open position;

FIG. 2 is a side perspective view of an end effector of the stapling device shown in FIG. 1 with a reload assembly of the end effector separated from an end effector frame;

FIG. 3 is an exploded side perspective view of a cartridge assembly of the reload assembly shown in FIG. 2;

FIG. 4 is an enlarged view of the indicated area of detail shown in FIG. 3 showing one side of a stopper of the cartridge assembly;

FIG. 5 is a side perspective view from an opposite side of the stopper shown in FIG. 4;

FIG. 6 is an enlarged view of the indicated area of detail shown in FIG. 3;

FIG. 7 is an enlarged view of the indicated area of detail shown in FIG. 3;

FIG. 8 is a side perspective view of the cartridge assembly shown in FIG. 3 assembled;

FIG. 9 is a side perspective view of the end effector shown in FIG. 2 with the reload assembly supported on the end effector frame and a cartridge body of the cartridge assembly shown in phantom;

FIG. 10 is an enlarged view of the indicated area of detail shown in FIG. 9;

FIG. 11 is a side cross-sectional cutaway view taken through the cartridge assembly with the cartridge assembly in an unclamped position;

FIG. 12 is a side cross-sectional cutaway view taken through the cartridge assembly during a firing stroke of the stapling device;

FIG. 13 is a side cross-sectional cutaway view taken through the cartridge assembly as a knife carrier of the cartridge assembly is retracted after the firing stroke of the stapling device;

FIG. 14 is a side cross-sectional cutaway view taken through the cartridge assembly with the knife carrier of the cartridge assembly in a post-fired, retracted, locked out position;

FIG. 15 is a cross-sectional view taken through an alternate version of the cartridge assembly of the stapling device shown in FIG. 1 with the cartridge assembly in a pre-fired unclamped position;

FIG. 16 is a side perspective view of a stopper of the cartridge assembly shown in FIG. 15;

FIG. 17 is a side cross-sectional view of the cartridge assembly shown in FIG. 15 with the knife carrier of the cartridge assembly in a post-fired, retracted, locked out position;

FIG. 18 is a side cross-sectional view taken through another alternate version of the cartridge assembly of the stapling device shown in FIG. 1 with the cartridge assembly in a pre-fired unclamped position;

FIG. 19 is a side perspective view of a stopper of the cartridge assembly shown in FIG. 18;

FIG. 20 is a side perspective view of an alternative version of the stopper shown in FIG. 18;

FIG. 21 is an enlarged view of the indicated area of detail shown in FIG. 18;

FIG. 22 is a side cross-sectional view taken through the cartridge assembly shown in FIG. 18 in the fired position; and

FIG. 23 is a side cross-sectional view taken through the cartridge assembly shown in FIG. 18 in the fired position with the knife assembly returned to its retracted position.

### DETAILED DESCRIPTION

The disclosed surgical stapling device will now be described in detail with reference to the drawings in which like reference numerals designate identical or corresponding elements in each of the several views. However, it is to be understood that the aspects of the disclosure are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the stapling device in its customary manner, while the term “distal” is used generally to refer to that portion of the device that is farther from the clinician during use of the stapling device in its customary manner. In addition, the term “clinician” is used generally to refer to medical personnel including doctors, nurses, and support personnel. Further, directional terms such as front, rear, upper, lower, top, bottom, and similar terms are used to assist in understanding the description and are not intended to limit the disclosure.

This disclosure is directed to a surgical stapling device that includes an end effector that has a cartridge assembly with a stopper of simple construction that prevents advancement of a pusher and a knife assembly within a cartridge body of the cartridge assembly until the stapling device is fired. This disclosure is also directed to a stapling device that includes a stopper of simple construction that prevents readvancement of the knife assembly of the cartridge assembly after the stapling device is fired.

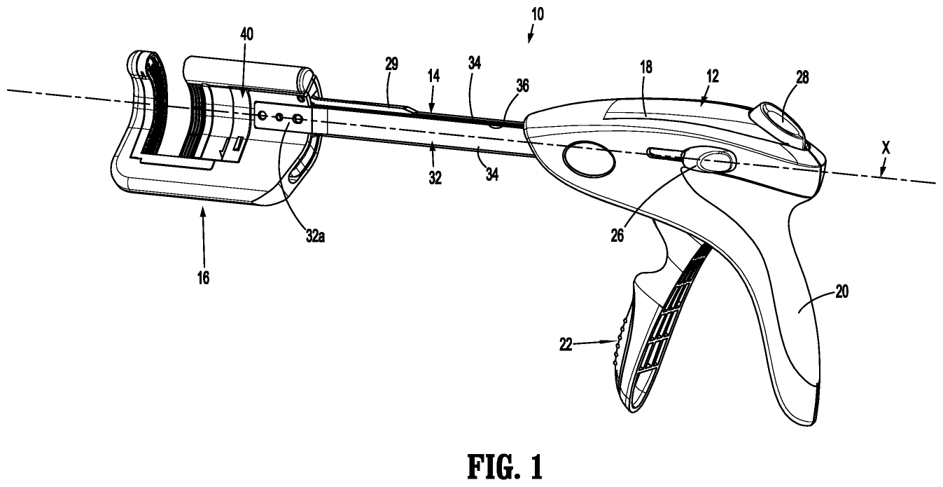

FIG. 1 illustrates a surgical stapling device according to aspects of the disclosure shown generally as stapling device 10. The stapling device 10 includes a handle assembly 12, an elongate body 14 that extends distally from the handle assembly 12, and an end effector 16 that is supported on a distal portion of the elongate body 14. The elongate body 14 defines a longitudinal axis “X”. The handle assembly 12 includes a housing 18 that defines a stationary handle 20 and supports a movable trigger 22. In aspects of the disclosure, the trigger 22 is supported by the housing 18 to pivot towards the stationary handle 20 between non-actuated and actuated positions to actuate the end effector 16, i.e., move the end effector 16 between open and clamped positions and fire the stapling device 10. The handle assembly 12 supports two buttons 26 (only one is shown), one button 26 positioned on each side of the housing 18, that are movable along the housing 18 to advance and retract an alignment pin pusher 29. The handle assembly 12 also includes a release button 28 that can be depressed to move the end effector 16 from the clamped position to the open position. For a more detailed description of a representative handle assembly 12, see, e.g., U.S. Pat. No. 6,817,508 (“the '508 patent”).

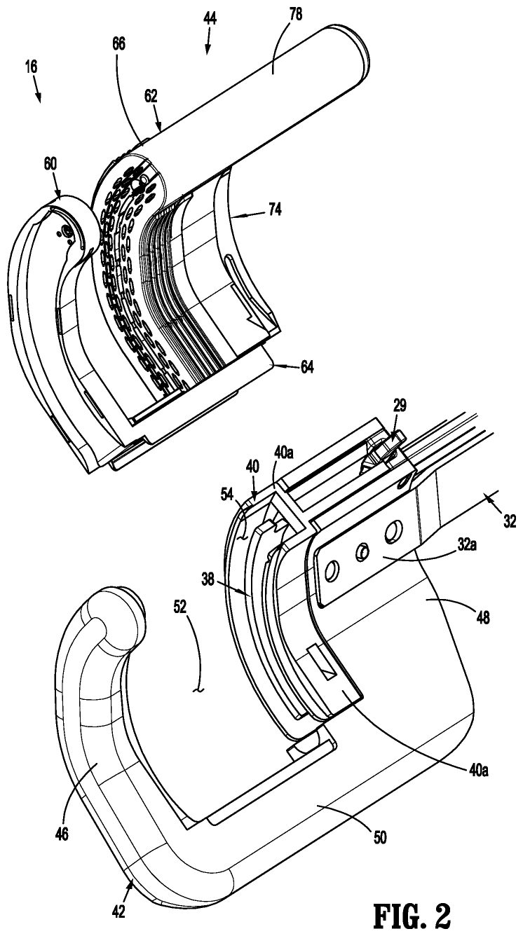

The stapling device 10 includes a frame 32 that extends from the handle assembly 12 and includes a distal portion 32a that supports the end effector 16. The frame 32 includes spaced frame members 34 that define a channel 36 that receives a thrust bar 38 (FIG. 2) and the alignment pin pusher 29. The thrust bar 38 (FIG. 2) is movable within the channel 36 between retracted and advanced positions in response to movement of the trigger 22 to actuate the stapling device 10. The channel 36 defined by the frame members 34 also receives a clamp slide assembly 40 that extends into the end effector 16. For a more detailed description of the interaction between the trigger 22, the thrust bar 38, and the clamp slide assembly 40, see the '508 patent.

FIG. 2 illustrates the end effector 16 which includes an end effector frame 42 and a reload assembly 44. The end effector frame 42 is secured to the distal portion 32a of the frame 32 of the stapling device 10 (FIG. 1) and includes a first transverse portion 46, a second transverse portion 48, and a longitudinal portion 50 that interconnects the first transverse portion 46 and the second transverse portion 48. The first and second transverse portions 46, 48 are spaced from each other to define a gap 52. In some aspects of the disclosure, the first and second transverse portions 46, 48 are curved along axes transverse to the longitudinal axis “X” of the elongate body 14 of the stapling device 10. In some aspects of the disclosure, the first and second transverse portions 46, 48 are formed from a plurality of linear portions, e.g., three, that are positioned at angles in relation to each other.

The clamp slide assembly 40 includes a proximal portion (not shown) that is coupled to the handle assembly 12 and a distal portion 40a (FIG. 2) that is received within the gap 52 defined between the first and second transverse portions 46, 48 of the end effector frame 42. The distal portion 40a of the clamp slide assembly 40 defines a cavity 54. The thrust bar 38 includes distal portion 38a that is received within the cavity 54 when the clamp slide assembly 40 and the thrust bar 38 are in retracted positions. The cavity 54 of the clamp slide assembly 40 receives the reload assembly 44 to releasably secure the reload assembly 44 to the end effector frame 42 and couple the thrust bar 38 to the reload assembly 44.

The reload assembly 44 (FIG. 2) includes an anvil assembly 60, a cartridge assembly 62, and a reload frame 64. The anvil assembly 60 and the cartridge assembly 62 are coupled to the reload frame 64 in a manner to facilitate movement of the cartridge assembly 62 in relation to the anvil assembly 60 between the open and clamped positions.

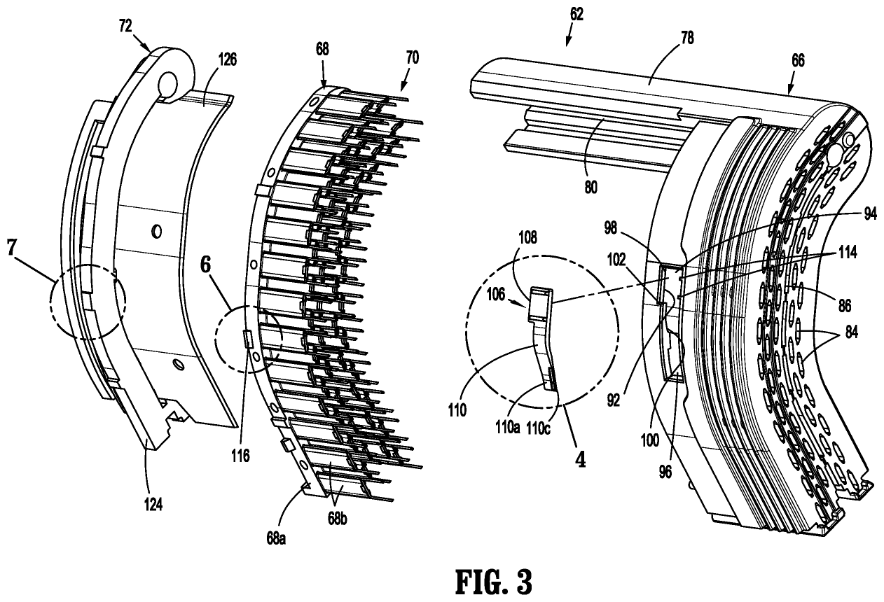

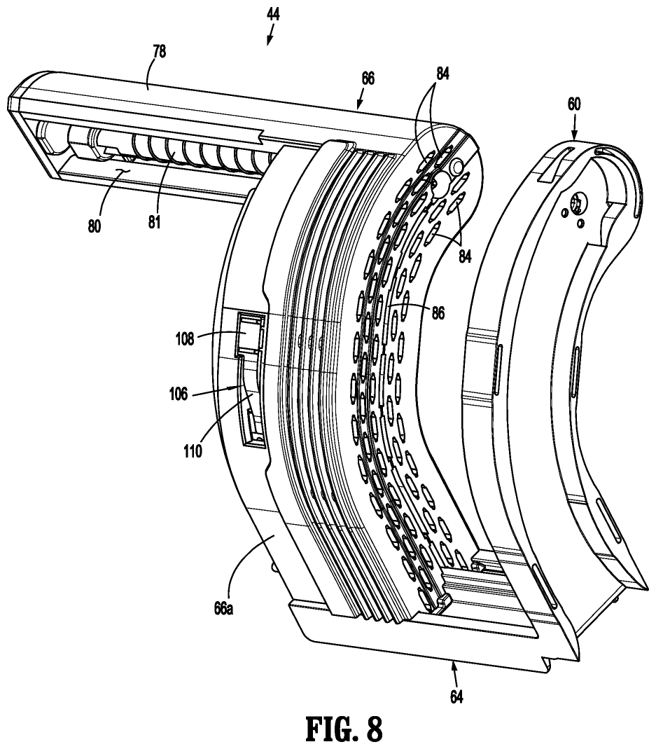

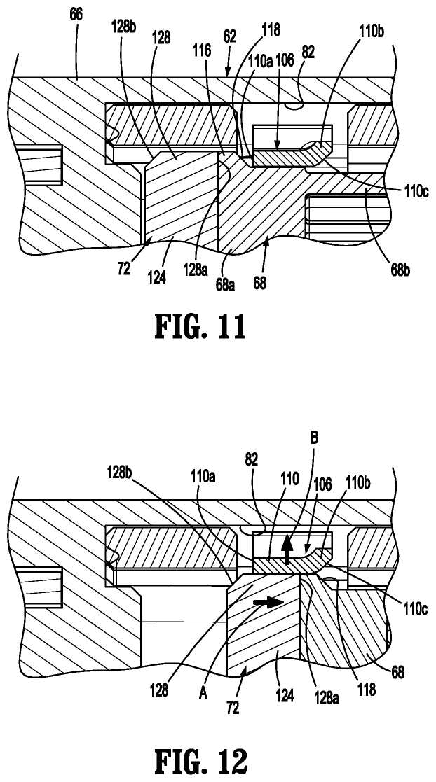

FIGS. 2 and 3 illustrate the cartridge assembly 62 which includes a cartridge body 66, a pusher 68 (FIG. 3), staples 70, and a knife assembly 72. A housing 74 (FIG. 2) is secured to a proximal portion of the cartridge body 66. The cartridge body 66 has a shape that corresponds to the shape of the first transverse portion 48 (FIG. 2) of the end effector frame 42 and includes a body portion 66a and a proximal extension 78 that is positioned on one side of the body portion 66a and defines a longitudinally extending bore 80 (FIG. 3) that receives an alignment pin assembly 81 (FIG. 8). The body portion 66a of the cartridge body 66 defines a cavity 82 (FIG. 11) that has an open proximal end, a plurality of staple receiving slots 84 that communicate with the cavity 82, and a knife slot 86 that also communicates with the cavity 82. In aspects of the disclosure, the staple receiving slots 84 are aligned in rows on opposite sides of the knife slot 86. Alternately, the staple receiving slots 84 may be formed in the body portion 66a of the cartridge body 66 in a variety of different configurations. Each of the staples 70 is received in a respective one of the staple receiving slots 84 of the cartridge body 66. The cavity 82 of the cartridge body 66 receives the pusher 68 and the knife assembly 72 and is dimensioned to allow movement of the pusher 68 and the knife assembly 72 within the body portion 66a between retracted and advanced positions. The body portion 66a has a side spaced from the proximal extension 78 that is configured to engage the reload frame 64 (FIG. 2) in interlocking fashion to slidably secure the cartridge body 66 to the reload frame 64.

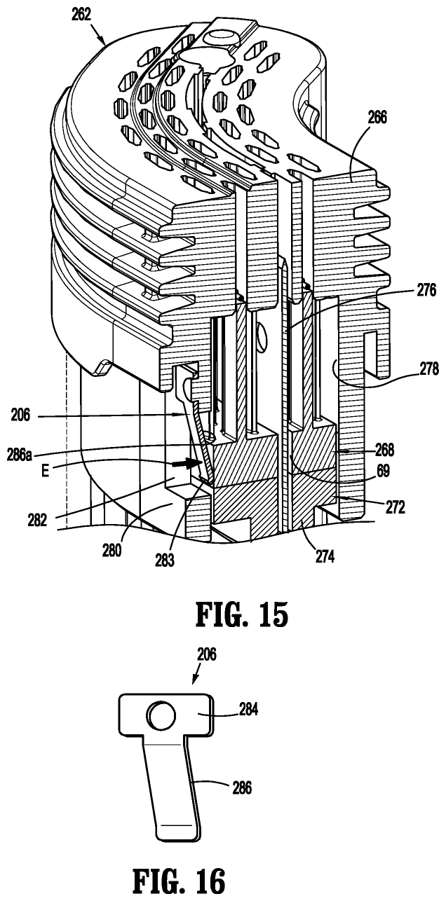

The pusher 68 is positioned within the cartridge body 66 distally of the knife assembly 72 and includes a base portion 68a and rows of fingers 68b (FIG. 3) that extend distally from the base portion 68a and are received within the staple receiving slots 84 of the cartridge body 66. The base portion 68a of the pusher 68 defines a knife slot 69 (FIG. 15) that extends through the base portion 68a of the pusher 68 and is positioned between the rows of fingers 68b. The knife assembly 72 is positioned in abutting relation to the pusher 68 such that movement of the knife assembly 72 within the cavity 82 of the cartridge body 66 causes movement of the pusher 68 within the cavity 82 to eject the staples 70 from the staple receiving slots 84.

The cartridge body 66 includes a side wall 90 that defines a recess 92. The recess 92 is defined by an inner support wall 94 that tapers inwardly towards an opening 96 in the side wall 90. The opening 96 communicates with the cavity 82 defined by the cartridge body 66. In aspects of the disclosure, the recess 92 has a first portion 98 and a second portion 100 that has a width that is smaller than the first portion 98 to define a shoulder 102 between the first portion 98 and the second portion 100. It is envisioned that the recess 92 can have a variety of configurations.

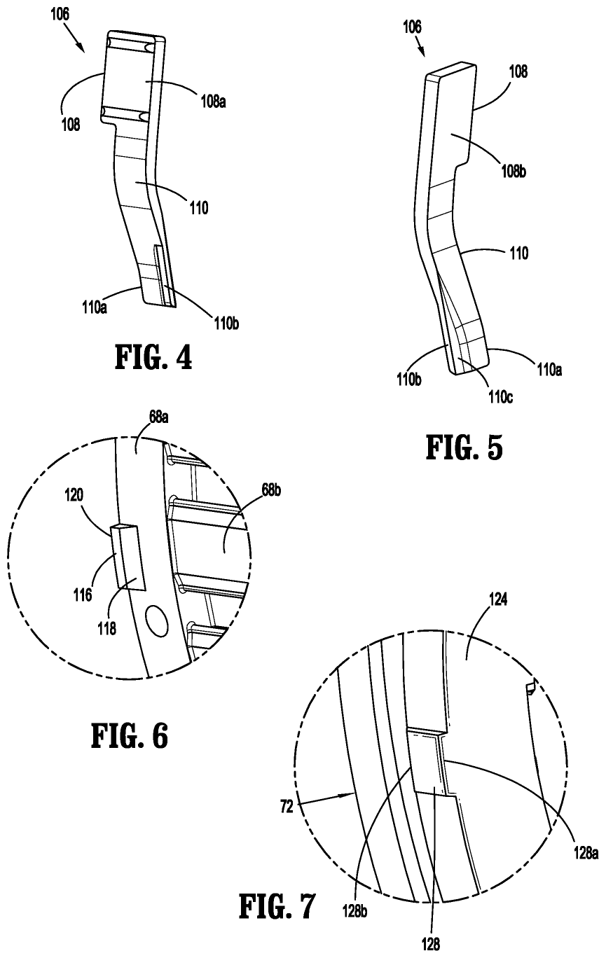

The recess 92 of the cartridge body 66 receives a stopper 106 which is illustrated in FIGS. 4 and 5. The stopper 106 includes a base portion 108 and a resilient arm 110 that extends transversely from the base portion 108 along the cartridge body 66. The resilient arm 110 includes a proximally facing stop surface 110a and a distally positioned curved rib 110b (FIG. 11) that are axially aligned with each other. The curved rib 110b is curved outwardly from the arm 110 as shown in FIG. 11 to define a distally facing cam surface 110c.

The base portion 108 of the stopper 106 has an outer surface 108a (FIG. 4) and an inner surface 108b (FIG. 5) and the arm 110 is angled inwardly from the base portion 108 towards the cavity 82 of the cartridge body 66. In aspects of the disclosure, the arm 110 of the stopper 106 has a curvature that corresponds to the curvature of the inner support surface 94 defining the recess 92 in the cartridge body 66. The arm 110 extends from the base portion 108 along the recess 92, through the opening 96 in the side wall 90 of the cartridge body 66, and into the cavity 82 of the cartridge body 66 such that the stop surface 110a of the arm 110 is positioned within the cavity 82 of the cartridge body 66.

In aspects of the disclosure, the base portion 108 of the stopper 106 is fixedly secured, e.g., press-fit, into the first portion 98 of the recess 92 of the cartridge body 66 such that the arm 110 of the stopper 106 is supported within the recess 92 of the cartridge body 66 in cantilevered fashion. The arm 110 is deformable from a first non-deformed position in which the stop surface 110a of the arm 110 is received within the cavity 82 of the cartridge body 66 and obstructs movement of the pusher 68 from its retracted position towards its advanced position, to a deformed position in which the stop surface 110a of the arm 110 is biased outwardly of the pusher 68 to allow for movement of the pusher 68 from its retracted position towards its advanced position. In this respect, the cartridge body 66 can include protrusions 114 that engage the base portion 108 of the stopper 106 to retain the stopper 106 within the recess 92. Alternately, the stopper 106 can be secured within the recess 92 of the cartridge body 66 using a variety of different securement techniques.

The base portion 68a of the pusher 68 supports, or is integrally formed with, an abutment member 116 which is illustrated in FIG. 6. The abutment member 116 is axially aligned with the stop surface 110a and the raised rib 110b of the stopper 106 and includes an angled or ramped distal surface 118 and an orthogonal proximal surface 120.

The knife assembly 72 includes a knife holder 124 and a cutting blade 126 that is secured to and extends distally from the knife holder 124. The cutting blade 126 extends through the knife slot 69 (FIG. 15) of the pusher 68 and is aligned with the knife slot 86 in the cartridge body 66. The knife holder 124 supports, or is integrally formed to include an engagement member 128 which is illustrated in FIG. 7. The engagement member 128 is axially aligned with the stop surface 110a and the raised rib 110b of the stopper 106 and includes a distally facing stop surface 128a and a proximally facing cam surface 128b. When the pusher 68 and the knife assembly 72 are positioned within the cavity 82 of the cartridge body 66 in their retracted positions, the abutment member 116 of the pusher 68 is aligned with and positioned distally of the engagement member 128 of the knife holder 124.

FIG. 8 illustrates the reload assembly 44 prior to loading of the reload assembly 42 onto the end effector frame 42 (FIG. 2) of the end effector 16 with the cartridge assembly 62 in a pre-fired condition. In the pre-fired condition, the stopper 106 is positioned such that the arm 110 extends through the opening 96 in the side wall 90 of the cartridge body 66 to position the stop surface 110a of the stopper 106 distally of the abutment member 116 of the pusher 68 (FIG. 10). In this position, the stop surface 110a of the stopper 106 prevents movement of the pusher from its retracted position towards its advanced position until a pre-determined force is applied to the pusher 68 in a distal direction that is sufficient to deform the arm 110 from its non-deformed position to its deformed position. In aspects of the disclosure, this force is generated via actuation of the movable handle 22 (FIG. 1) although other powered and/or robotically controlled actuation devices are also envisioned to advance the pusher 68 to reach the pre-determined force. As described above, the knife holder 124 is positioned proximally of and in abutting relation with the pusher 68. As such, the stop surface 110a of the stopper 106 also prevents advancement of the knife holder 124 within the cavity 82 of the cartridge body 66 until the pre-determined force is applied to the pusher 68.

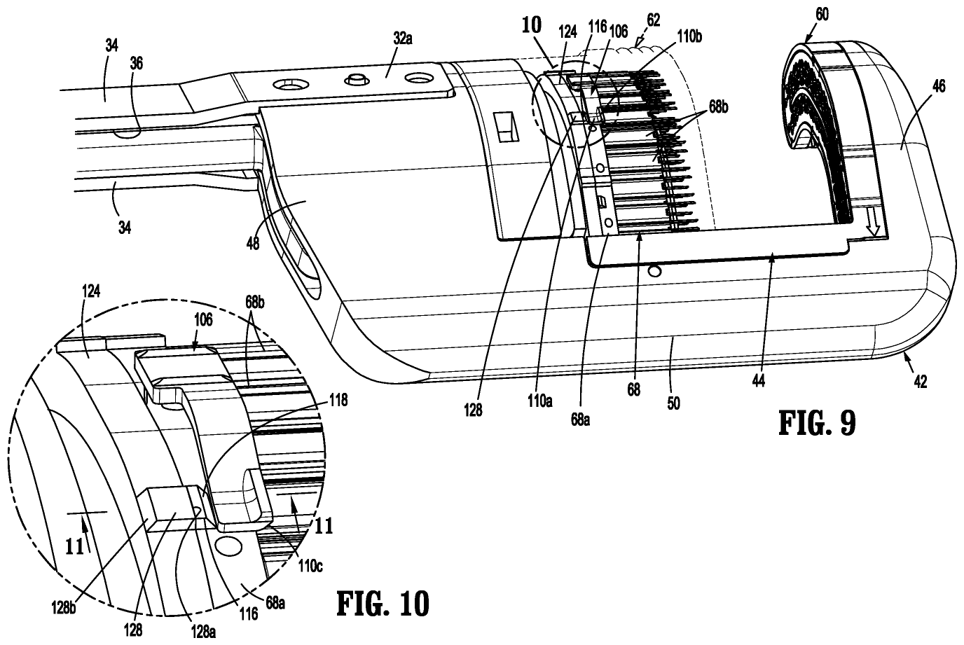

FIGS. 9 and 10 illustrate the reload assembly 44 after it has been loaded onto the end effector frame 42 (FIG. 2) of the end effector 16. When the reload assembly 44 is loaded onto the end effector frame 42 of the end effector 16, the cartridge assembly 62 is received within the cavity 54 (FIG. 2) of the clamp slide assembly 40. As the cartridge assembly 62 is received within the cavity 54 of the clamp slide assembly 40, the thrust bar 38 of the stapling device 10 (FIG. 1) engages the knife holder 124 of the knife assembly 72. With the cartridge assembly 62 received within the cavity 54 of the clamp slide assembly 40, the movable trigger 22 can be actuated to move the cartridge assembly 62 in relation to the anvil assembly 60 between the open and approximated positions. Once in the approximated position, the movable trigger 22 can be actuated again to advance the thrust bar 38 from a retracted position to an advanced position. As described above, the thrust bar 38 is coupled to the knife holder 124. Thus, movement of the thrust bar 38 from its retracted position towards its advanced position moves the knife assembly 72 and the pusher 68 from their retracted positions to their advanced positions. For a more detailed description of the interaction between the clamp slide assembly 40, the thrust bar 38, the knife assembly 72, and the pusher 68, see the '508 patent.

FIGS. 11 and 12 illustrate interaction of the stopper 106, the pusher 68, and the knife assembly 72 as the stapling device 10 (FIG. 1) is fired to eject staples 70 (FIG. 3) from the cartridge body 66. As described above, prior to firing of the stapling device 10 (FIG. 1), the stop surface 110a of the stopper 106 is positioned distally of the ramped distal surface 118 of the abutment member 116 of the pusher 68 to retain the pusher 68 in its retracted position (FIG. 11). When the thrust bar 38 (FIG. 2) is advanced via actuation of the movable trigger 22, the knife holder 124 is advanced in the direction of arrow “A” in FIG. 12 to advance the pusher 68 in the direction of arrow “A”. As the pusher 68 is advanced, the ramped distal surface 118 of the abutment member 116 on the pusher 68 engages and deforms the arm 110 of the stopper 106 outwardly in the direction of arrow “B” in FIG. 12 to move the stop surface 110a outwardly of the pusher 68 from its first position towards its second position to allow the pusher 68 to move towards its advanced position. Engagement between the abutment member 116 of the pusher 68 and the stopper 106 allows the engagement member 128 of the knife holder 124 to move under the stop surface 110a of the stopper 106 to allow the knife holder 124 to move distally past the stop surface 110a of the stopper 106 to its advanced position. When the pusher 68 moves to its advanced position, the fingers 68b (FIG. 11) of the pusher 68 move through the staple receiving slots 84 in the cartridge body 66 to eject the staples 70 from the cartridge body 66. In addition, as the knife holder 124 is advanced towards its advanced position, the cutting blade 126 of the knife assembly 72 is advanced through the knife slot 86 (FIG. 3) of the cartridge body 66 to cut tissue clamped between the cartridge and anvil assemblies 60 and 62 (FIG. 2)

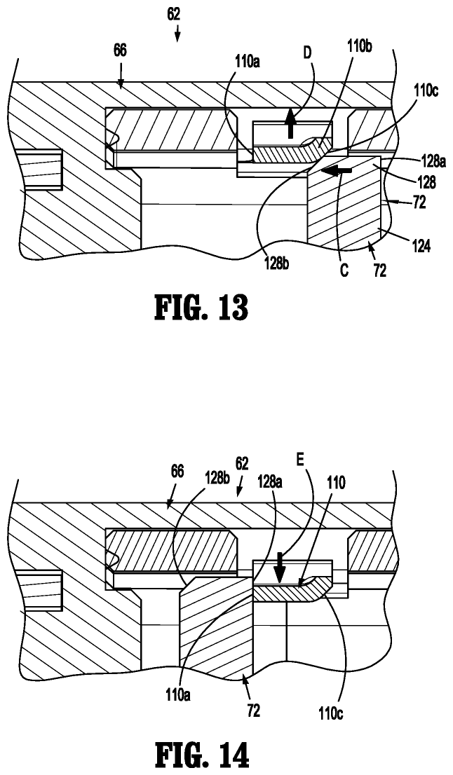

FIGS. 13 and 14 illustrate the cartridge assembly 62 as the knife assembly 72 is moved from its advanced position back to its retracted position after the stapling device 10 (FIG. 1) is fired. When the thrust bar 38 (FIG. 2) is retracted after the stapling device 10 is fired, the thrust bar 38 returns the knife assembly 72 in the direction of arrow “C” in FIG. 13 to its retracted position. It is noted that the knife holder 124 of the knife assembly 72 is in abutting relation with the pusher 68. As such, the pusher 68 is not retracted with the knife assembly 72. As the knife assembly 72 is retracted with the thrust bar 38, the angled proximal surface 128b of the engagement member 128 of the knife holder 124 engages the distally facing cam surface 110c of the stopper 106. When this occurs, continued retraction of the knife holder 124 cams the arm 110 of the stopper 106 outwardly in the direction of arrow “D” in FIG. 13 to allow the knife holder 124 to move past the stopper 106 and return to its retracted position to return the cutting blade 126 to its retracted position. When the knife holder 124 is in its retracted position, the cutting blade 126 of the knife assembly 72 is recessed and shielded within the cartridge body 66 of the cartridge assembly 62.

When the knife holder 124 moves past the arm 110 of the stopper 106, the arm 110 of the stopper 106 which is resilient returns to its non-deformed position in the direction of arrow “E” in FIG. 14. In its non-deformed position, the stop surface 110a of the stopper 110 is aligned with the stop surface 128a of the engagement member 128 of the knife holder 124 to prevent readvancement of the knife holder 124 within the cavity 82 of the cartridge body 66 and retain the cutting blade 126 in a shielded position within the cartridge body 66 of the cartridge assembly 62.

FIGS. 15-17 illustrate an alternate version of a cartridge assembly and stopper according to aspects of the disclosure shown generally as cartridge assembly 262 and stopper 206. The cartridge assembly 262 is substantially as described above regarding cartridge assembly 62 (FIG. 3) and includes a cartridge body 266, a pusher 268, and a knife assembly 272 that includes a knife holder 274 and a cutting blade 276. The cartridge body 266 defines a cavity 278 and includes a side wall 280 that defines a recess 282 and an opening 283 that communicates with the cavity 278. The cartridge assembly 262 functions in manner substantially similar to the cartridge assembly 62 described above and will not be described in further detail herein.

The stopper 206 is secured within the recess 282 in the side wall 280 of the cartridge body 266 and includes a base portion 284 and a resilient arm 286 that is secured to or integrally formed with the base portion 284. The resilient arm 286 extends proximally and inwardly from the base portion 284 at an angle through the opening 283 in the side wall 280 of the cartridge body 266 to a position to engage the pusher 268 (when the pusher 268 is in its retracted position). The resilient arm 286 includes a distally facing angled cam surface 286a. When the pusher 268 is in its retracted position, the resilient arm 286 is deformed outwardly and applies a frictional force on the pusher 268 in the direction of arrow “E” in FIG. 15 to retain the pusher 268 in its retracted position. As described above regarding the cartridge assembly 62, the retaining force applied to the pusher 268 by the resilient arm 286 of the stopper 206 can be overcome by applying a predetermined force in a distal direction to advance the pusher 268 from its retracted position towards its advanced position. In aspects of the disclosure, this pre-determined force is generated via actuation of the movable handle 22 (FIG. 1) although other powered and robotically controlled actuation devices are also envisioned to apply the pre-determined force to pusher 268.

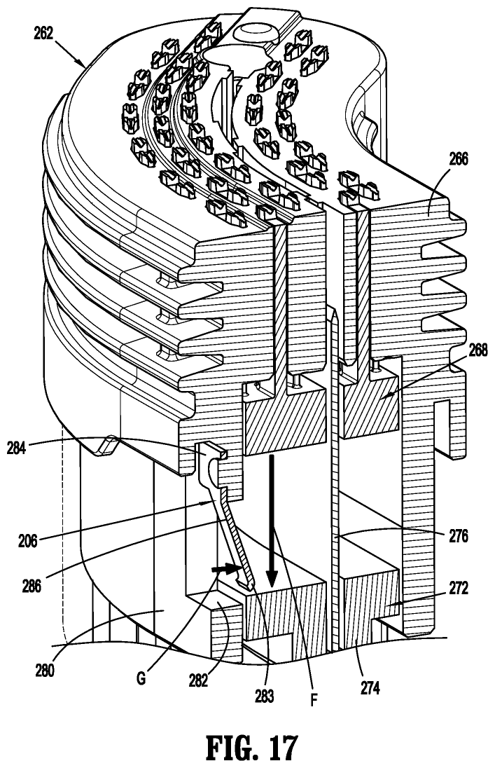

FIG. 17 illustrates the cartridge assembly 262 after the cartridge assembly 262 is fired and the knife holder 274 is retracted. As illustrated, the knife holder 274 is retracted independently of the pusher 268 in the direction of arrow “F”. When the knife holder 274 is retracted to a position to engage the distally facing angled cam surface 286a of the resilient arm 286 of the stopper 206, the resilient arm 286 is cammed outwardly to allow the knife holder 274 to move past the resilient arm 286 of the stopper 206. When the knife holder 274 passes the stopper 206, the resilient arm 286 returns to its non-deformed position in the direction of arrow “G”. In its non-deformed position, a stop surface 286a of the arm 286 of the stopper 206 is positioned to obstruct readvancement of the knife holder 274 to retain the knife holder 274 and the cutting blade 276 in their retracted positions. When the knife assembly 272 is in its retracted position, the cutting blade 276 of the knife assembly 272 is recessed and shielded within the cartridge body 266 of the cartridge assembly 262.

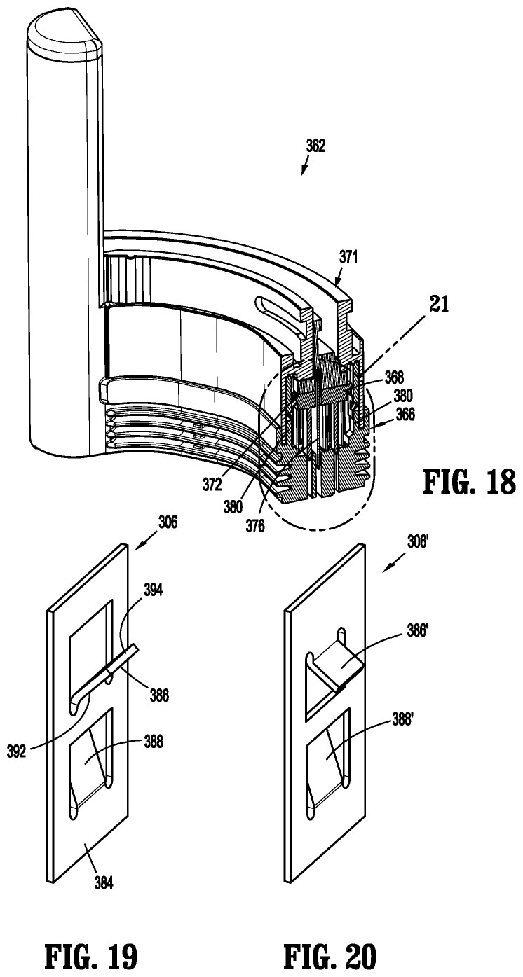

FIGS. 18-23 illustrate an alternate version of a cartridge assembly and stopper according to aspects of the disclosure shown generally as cartridge assembly 362 and stopper 306. The cartridge assembly 362 is substantially as described above regarding cartridge assemblies 62 and 262 and includes a cartridge body 366, a pusher 368, a housing 371, and a knife assembly 372 that includes a knife holder 374 and a cutting blade 376. The cartridge body 366 includes side walls 380 that include inner surfaces 382 that define a cavity 378. The pusher 368 and the knife assembly 372 are received within the cavity 378 and are moveable between retracted and advanced positions within the cavity 378 to eject staples (not shown) from the cartridge body 366. The cartridge assembly 362 functions in manner substantially similar to the cartridge assemblies 62 and 262 described above and will not be described in further detail herein.

The stopper 306 includes a body 384 (FIG. 19) that has a plate-like configuration and includes inwardly and outwardly extending angled resilient arms or tangs 386 and 388. In aspects of the disclosure, the inwardly extending angled arm 386 is positioned distally of the outwardly extending angled arm 388. In an alternative version of the stopper shown generally as stopper 306′ (FIG. 20), the inwardly extending angled arm 386′ is positioned proximally of the outwardly extending angled arm 388′. The stopper 306 is secured to the inner surface 382 of one, or both, of the side walls 380 of the cartridge body 366. In aspects of the disclosure, the outwardly extending angled arm 388 is received within an opening 390 formed in the side wall 380 of the cartridge body 366 to secure the stopper 306 to the inner surface 382 of the side wall 380. It is envisioned that the stopper 306 (and 306′) could be secured to the inner surface 382 using other securement techniques and need not include the outwardly extending angled arm 388. The inwardly extending angled arm 386 extends into the cavity 378 in a proximal direction and includes an angled distally facing cam surface 392 and a proximal stop surface 394.

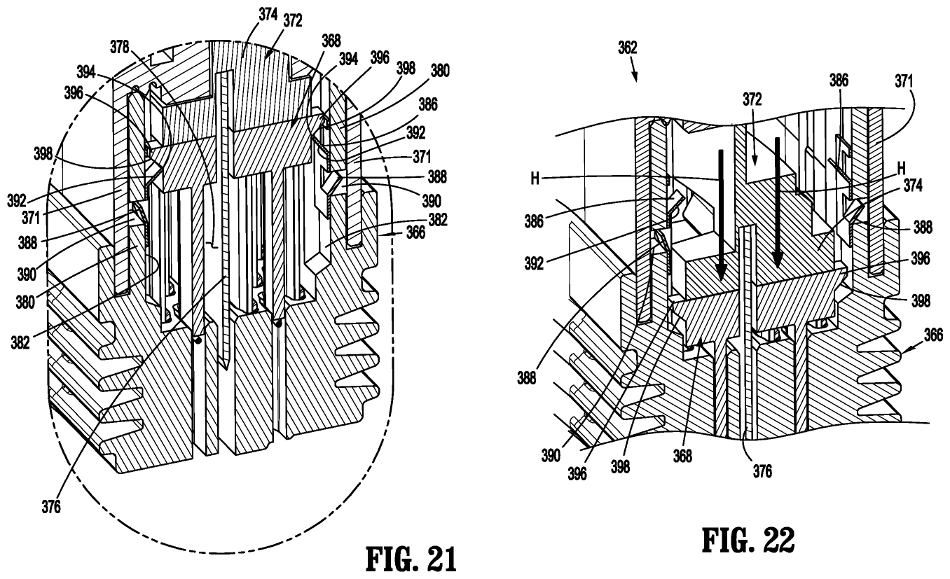

The pusher 368 includes an abutment member 396 that has a ramped distal surface 398. When the pusher 368 and the knife assembly 372 are in their retracted positions, the proximal stop surface 394 of the inwardly extending angled arm 386 is engaged with the ramped distal surface 398 of the abutment member 396 of the pusher 368 to prevent advancement of the pusher 368 and the knife assembly 372 within the cavity 378 of the cartridge body 366 until a predetermined force is applied to the pusher 368 (FIG. 21). In aspects of the disclosure, this pre-determined force is generated via actuation of the movable handle 22 (FIG. 1) although other powered and/or robotically controlled actuation devices are also envisioned to apply the pre-determined force to the pusher 368.

FIG. 22 illustrates the cartridge assembly 362 as the knife assembly 372 and the pusher 368 are moved from their retracted positions to their advanced positions in the direction indicated by arrows “H”. As the knife assembly 372 and the pusher 368 are moved towards their advanced positions, the angled arm 386 of the stopper 306 is deformed and moves over the ramped distal surface 398 of the abutment member 396 of the pusher 368 to allow the pusher 368 and the knife assembly 372 to move distally past the angled arm 386 to eject staples (not shown) from the cartridge body 366 and advance the cutting blade 376 from the cartridge body 366 of the cartridge assembly 362.

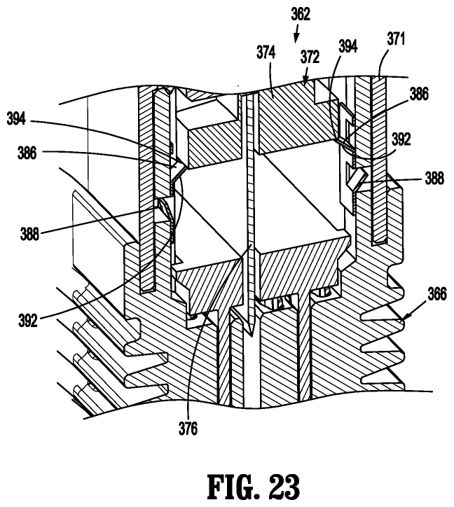

FIG. 23 illustrates the cartridge assembly 362 as the knife assembly 372 is moved back to its retracted position after the stapling device 10 (FIG. 1) is fired. As the knife assembly 372 is retracted, a proximal surface of the knife holder 374 engages the angled distally facing cam surface 392 of the angled arm 386 to deform the angled arm 386 outwardly and allow the knife holder 374 to move to the retracted position and withdraw the cutting blade 376 into the cartridge body 366. When the knife holder 374 passes over the angled arm 386, the angled arm 386 returns to its non-deformed position. In its non-deformed position, the proximal stop surface 394 of the angled arm 386 is positioned within the cavity 378 to obstruct readvancement of the knife assembly 372.

In aspects of the disclosure, the stoppers 106, 206, and 306 are formed from a resilient material such as spring steel. Alternately, other resilient materials including metal and plastics can be used to form the stoppers 106, 206, and 306.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A cartridge assembly comprising:
a cartridge body defining a cavity, a knife slot that communicates with the cavity, and a plurality of staple receiving slots, the staple receiving slots positioned on opposite sides of the knife slot;
a staple supported within each of the staple receiving slots of the cartridge body;
a pusher supported within the cavity of the cartridge body, the pusher including a base portion and fingers extending distally from the base portion into the staple receiving slots, the base portion defining a knife receiving slot;
a knife assembly including a knife holder and a cutting blade extending distally from the knife holder, the cutting blade extending through the knife receiving slot of the base portion of the pusher and movable from a retracted position to an advanced position to move the cutting blade through the knife slot of the cartridge body; and
a resilient stopper supported on the cartridge body, the resilient stopper deformable from a first position in which the resilient stopper extends into the cavity of the cartridge body to obstruct movement of the pusher and the knife assembly from their retracted positions to their advanced positions to a second position allowing movement of the pusher and the knife assembly from their retracted positions to their advanced positions.

2. The cartridge assembly of claim 1, wherein the cartridge body includes a side wall that defines a recess and an opening that communicates with the recess and the cavity, the resilient stopper positioned within the recess.

3. The cartridge assembly of claim 2, wherein in the first position, the stopper is in a non-deformed position.

4. The cartridge assembly of claim 2, wherein the pusher includes an abutment member that is positioned to engage the stopper to deform the stopper from the first position to the second position.

5. The cartridge assembly of claim 4, wherein the abutment member includes a ramped distal surface, the ramped distal surface positioned to engage the stopper to move the stopper from the first position to the second position.

6. The cartridge assembly of claim 5, wherein the stopper includes a base portion and an arm, the base position fixedly secured within the recess and the arm coupled to the base portion in cantilevered fashion.

7. The cartridge assembly of claim 6, wherein the arm has a proximally facing stop surface that is positioned within the cavity of the cartridge body when the stopper is in the first position, the proximally facing stop surface being axially aligned with the abutment member of the pusher.

8. The cartridge assembly of claim 7, wherein the knife holder includes an engagement member that is axially aligned with the abutment member of the pusher and the proximally facing stop surface of the arm.

9. The cartridge assembly of claim 8, wherein the arm includes a distally facing cam surface, the engagement member being axially aligned with the distally facing cam surface and movable into engagement with the distally facing cam surface when the knife holder is retracted to move the stopper to the second position and allow the knife holder to move from the advanced position back to the retracted position.

10. The cartridge assembly of claim 9, wherein the stopper moves back to the first position after the knife holder moves back to its retracted position to prevent readvancement of the knife holder.

11. The cartridge assembly of claim 1, wherein the stopper is positioned to engage the pusher to frictionally retain the pusher in its retracted position.

12. The cartridge assembly of claim 11, wherein the stopper includes a resilient arm that includes a distally facing cam surface and a proximal stop surface.

13. The cartridge assembly of claim 12, wherein the knife holder is movable into engagement with the distally facing cam surface of the resilient arm as the knife holder is moved from its advanced position towards its retracted position to move the resilient arm of the stopper from the first position to the second position and allow the knife holder to return to its retracted position.

14. The cartridge assembly of claim 13, wherein the resilient arm of the stopper moves back to the first position after the knife holder moves back to its retracted position, and wherein the proximal stop surface of the resilient arm of the stopper is positioned to prevent readvancement of the knife holder when the stopper is in the first position.

15. The cartridge assembly of claim 1, wherein the stopper is secured to an inner surface of the side wall of the cartridge body and includes a resilient arm that is angled into the cavity in a proximal direction, the resilient arm of the stopper movable from the first position to the second position in response to movement of the pusher from its retracted position towards its advanced position.

16. The cartridge assembly of claim 15, wherein the pusher includes an abutment member having a ramped distal surface that is positioned to engage the resilient arm to move the resilient arm from the first position to the second position.

17. A surgical stapling device comprising:
an elongate body having a proximal portion and a distal portion;
an end effector supported on the distal portion of the elongate body, the end effector including:
an end effector frame; and
a reload assembly including:
a reload frame;
an anvil assembly supported on the reload frame; and
a cartridge assembly supported on the reload frame, the cartridge assembly including:
a cartridge body defining a cavity, a knife slot that communicates with the cavity, and a plurality of staple receiving slots, the staple receiving slots positioned on opposite sides of the knife slot;
a staple supported within each of the staple receiving slots of the cartridge body;
a pusher supported within the cavity of the cartridge body, the pusher including a base portion and fingers extending distally from the base portion into the staple receiving slots, the base portion defining a knife receiving slot;
a knife assembly including a knife holder and a cutting blade extending distally from the knife holder, the cutting blade extending through the knife receiving slot of the base portion of the pusher and movable from a retracted position to an advanced position to move the cutting blade through the knife slot of the cartridge body; and
a resilient stopper supported on the cartridge body, the resilient stopper deformable between a first position extending into the cavity of the cartridge body to obstruct movement of the pusher and the knife assembly from their retracted positions to their advanced positions and a second position allowing movement of the pusher and the knife assembly from their retracted positions to their advanced positions.

18. The stapling device of claim 17, further including a clamp slide assembly and a thrust bar, the clamp slide assembly defining a cavity that removably receives the cartridge assembly of the end effector, the clamp slide assembly movable between retracted and advanced positions to move the cartridge assembly in relation to the anvil assembly between open and clamped positions.

19. The stapling device of claim 18, wherein the thrust bar is adapted to releasably engage the knife holder of the cartridge assembly, the thrust bar movable from a retracted position to an advanced position to advance the pusher and eject the staples from the cartridge body of the cartridge assembly.

20. A cartridge assembly comprising:
a cartridge body defining a cavity, a knife slot that communicates with the cavity, and a plurality of staple receiving slots, the staple receiving slots positioned on opposite sides of the knife slot;
a staple supported within each of the staple receiving slots of the cartridge body;
a pusher supported within the cavity of the cartridge body and defining a knife receiving slot;
a knife assembly including a knife holder and a cutting blade extending distally from the knife holder, the cutting blade extending through the knife receiving slot of the and movable from a retracted position to an advanced position to move the cutting blade through the knife slot of the cartridge body; and
a stopper supported on the cartridge body, the stopper movable between a first position extending into the cavity of the cartridge body to obstruct movement of the pusher and the knife assembly from their retracted positions to their advanced positions and a second position allowing movement of the pusher and the knife assembly from their retracted positions to their advanced positions.
